(19) United States
(12) Patent Application Publication
(10) Pub. No.: US 2010/0059054 A1
(43) Pub. Date: Mar. 11, 2010

(54) DRY-STEAM INHALER

(76) Inventor: Yanis Lenggenhager, Neuchatel (CH)

Correspondence Address: YOUNG & THOMPSON, 209 Madison Street, Suite 500, Alexandria, VA, 22314, US

(21) Appl. No.: 12/447,288

(22) PCT Filed: Oct. 5, 2007
(86) PCT No.: PCT/EP2007/060621

§ 371 Date: Apr. 27, 2009

(30) Foreign Application Priority Data
Oct. 25, 2006 (EP) 06122902.7

(51) Int. Cl. A61M 16/10

(52) U.S. Cl. 128/203.26

(57) ABSTRACT

A dry-steam inhaler permits extraction of the active principles of plants or oils, especially of medicinal plants and oils, by heating, more particularly by using a flame as the source of heat. The inhaler includes a heating element (1) and an aspiration device, the heating element (1) having a base (3) and side walls (4) that project from the base (3) and delimit an aspiration chamber (5) communicating with the aspiration device, the base (3) of the heating element (1) being traversed by at least one air aspiration channel (6), of which one end (6a) opens out on the side walls (4) of the heating element (1), and of which the other end (6b) opens out in the aspiration chamber (5).

### TECHNICAL FIELD

The present invention concerns a dry-steam inhaler, permitting extraction of the active principles of plants or oils, especially of medicinal plants and oils, by heating. More particularly, the invention concerns a dry-steam inhaler using a flame, for example from a lighter, as the source of heat.

### BACKGROUND OF THE INVENTION

A dry-steam inhaler is an apparatus with which one can heat dry or fresh plants, oils or other plant-based preparations, or even medications intended to be vaporized, so as to extract the active principle in the form of steam, in order to directly inhale it, by providing heat without, however, burning them. The temperature at which the active principle is extracted is between 30° C. and 300° C. depending on the product, generally around 180° C. An inhaler comprises aspiration means to enable inhalation of the useful substances of a plant without absorbing other undesired substances such as carbon dioxide and nitric gases. An inhaler is particularly effective during therapeutic treatment and for use in aromatherapy or phytotherapy.

The inhalers known at this time are, for the most part, tabletop apparatuses comprising electric heating devices, which as a result require electrical power to operate. These apparatuses therefore present the drawback of not being easily portable. Furthermore, their design is sophisticated, making them costly. Also known are inhalers with a simpler design which, for example, take the form of a glass bowl heated using a lighter. This type of inhaler is therefore particularly fragile. There are also inhalers, such as those described in patent application WO 03/082031, whereof the base is traversed by transverse channels. These inhalers must then comprise thick bases in order to ensure good heating of the aspirated air. In general, heating the substance sufficiently to allow the extraction of the active principle, without burning the substance or having to reheat the substance at such a frequency that the use of the apparatus becomes inconvenient, is particularly delicate with a portable inhaler.

The inhalers must be differentiated from simple diffusers, which do not comprise aspiration means. For example, a diffuser of this type is described in document U.S. Pat. No. 2,591,818. According to one of the proposed embodiments, the diffuser comprises a grid (21) constituting a joule effect heating element. This grid is pierced, in its thickness, with transverse openings, so as to be able to be traversed by the air coming from outside the vaporizing device. However, this current of air is very regular and is not related to aspiration by the user. Furthermore, the fact that the heating element is an electric resistance heater means that a vaporizer of this type does not have the problem of the arriving air having to be at a good temperature, nor the problem of cooling the heating element.

### BRIEF DESCRIPTION OF THE INVENTION

The aim of the present invention is therefore to offset these various drawbacks by proposing a dry-steam inhaler operating without electricity and which has a reduced format while also having a solid and economical, portable design that is easy-to-use, fully cleanable and able to be sterilized and guarantees optimal heating of the product while, however, avoiding its combustion.

To this end, and according to the present invention, proposed is a dry-steam inhaler, comprising a heating element and an aspiration device, characterized in that the heating element comprises a base and side walls that project from this base and delimit an aspiration chamber communicating with the aspiration device, the base of the heating element being traversed by at least one air aspiration channel whereof one end opens out on the side walls of the heating element and the other end of which opens out in the aspiration chamber. Thanks to this particularly interesting design, the aspiration channel offers a sufficiently long path for the aspirated air to enable it to heat in contact with the heating element. The substance is therefore only slightly cooled, which allows prolonged inhalation before having to heat the heating element again.

Particularly advantageously, the base of the heating element can comprise four air aspiration channels arranged in a cross, their ends which open out in the aspiration chamber meeting at the center of said aspiration chamber.

Preferably, the air aspiration channels are arranged in the base of the heating element parallel to said base.

In one preferred embodiment, the side walls of the heating element comprise a lower extension which projects from the base on the opposite side from the aspiration chamber and defines, with the base, a heating crucible.

In order to avoid combustion and improve heat conductivity up to the substance, the dimensions of the heating element are optimized. In particular, the heating element is cylindrical in shape and the air aspiration channel can have a diameter smaller than or equal to 2 mm and be arranged in the base of the heating element parallel to the heating crucible and 1 mm or less from it, and parallel to the aspiration chamber and 1 mm or less from it.

Advantageously, the base of the heating element can comprise, in its center, a hollow form in which the ends of the air aspiration channels open.

So as to improve heat conductivity, the heating element is made in a heat conducting material such as an aluminum-based material or another material which has sufficient heat conductivity and capacity and is treated against corrosion. This makes it possible to further improve the duration of the inhalations without having to reheat the heating element.

As for the aspiration device, it comprises an aspiration cone arranged in order to cooperate with the heating element and an aspiration straw.

Lastly, one can provide a thermal insulator arranged around the heating element and serving as protection.

The inhaler according to the invention is designed more particularly to be heated using a flame, placed under the heating crucible.

### BRIEF DESCRIPTION OF THE DRAWINGS

Other advantages and characteristics will better emerge from the description which follows, of a system according to the invention, provided as a non-limiting example, in reference to the appended drawings in which:

FIG. 1 is a top view of the heating element of the inhaler according to the invention,

FIG. 2 is a cross-sectional view along the line A-A of FIG. 1,

FIG. 3 is a side view of the aspiration device of the inhaler according to the invention, and

FIG. 4 is a top view of the aspiration device.

### DETAILED DESCRIPTION OF THE INVENTION

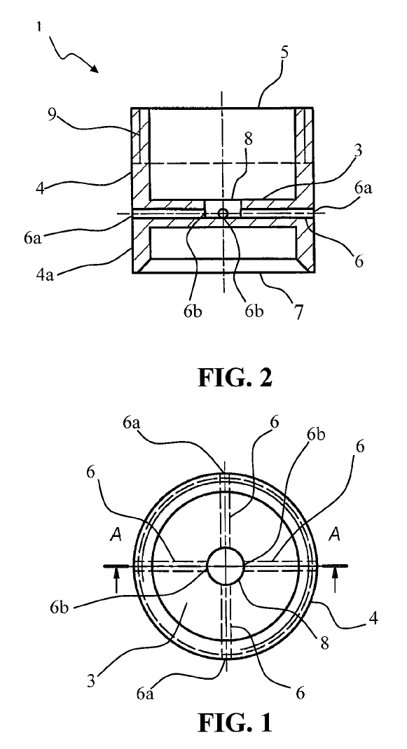

The dry-steam inhaler according to the invention comprises a heating element 1 and an aspiration device 2. In reference to FIGS. 1 and 2, the heating element 1 is cylindrical in shape. It is made in a single piece in aluminum. It is quite clear that one can use other shapes or other heat conducting materials without going beyond the scope of the present invention. The heating element 1 comprises a base 3 and side walls 4 which project from the base 3 and delimit an aspiration chamber 5 communicating with the aspiration device 2.

The side walls 4 of the heating element 1 comprise a lower extension 4a which projects from the base 3 on the opposite side from the aspiration chamber 5 and delimiting, with said base 3, a heating crucible 7.

According to the invention, the base 3 of the heating element 1 is traversed by four air aspiration channels 6, each with one of their ends 6a opening out on the side walls 4 of the heating element 1 and the other end 6b opening out in the aspiration chamber 5.

The four air aspiration channels 6 are arranged in a cross in the base 3 of the heating element 5 perpendicular to the side walls 4, said base 3 comprising, in its center, a hollow form 8 in which the ends 6b of the air aspiration channels 6 open and meet.

The dimensions of the cylindrical heating element 1, optimized to avoid combustion and improve heat conductivity up to the substance, are such that the inner diameter of the heating element is between 10 and 18 mm, preferably substantially equal to 16 mm, for a height between 10 and 20 mm, preferably substantially equal to 18 mm, and the thickness of the walls is between 1 and 3 mm, preferably substantially equal to 2 mm. The base 3 of the heating element 1 preferably has a thickness substantially equal to 3 mm and is placed such that the heating crucible 7 has a height preferably substantially equal to 3 mm and the aspiration chamber has a height preferably substantially equal to 10 mm. The air aspiration channels 6 each have a diameter smaller than 3 mm, preferably smaller than 2 mm, advantageously smaller than or equal to 1 mm and are arranged in the base 3 of the heating element 1, parallel to the heating crucible 7 and substantially 1 mm or less from it, and parallel to the aspiration chamber 5 and substantially 1 mm or less from it. It is quite clear that the air aspiration channels can also be arranged obliquely between the side walls 4 and the aspiration chamber 5, the main thing being to provide a sufficient journey for the aspirated air when it traverses the heating element so as to heat itself in contact with the heating element in order to reach the aspiration chamber hot.

The heating element 1 can comprise one or several grids (not shown) arranged in the aspiration chamber 5 on which the substance is placed or in order to distribute oils by capillarity. An additional grid is arranged at the inlet of the aspiration device in order to avoid particles of the element to be heated reaching the user.

The heating element 1 comprises, on its side walls 4, a threading 9 allowing fastening screwing of the aspiration device 2.

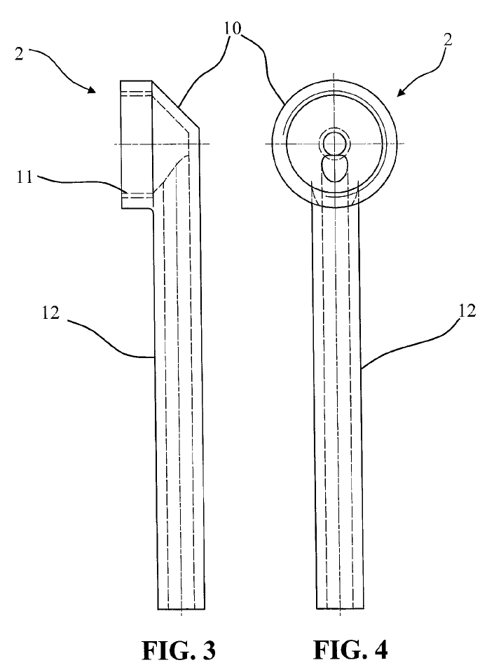

In reference to FIGS. 3 and 4, the aspiration device 2 comprises an aspiration cone 10 with dimensions appropriate to be assembled to the heating element 1. To this end, the inner wall of the aspiration cone 10 comprises a threading 11 corresponding to the threading 9 of the heating element 1 in order to ensure the assembly of two pieces by screwing. The aspiration cone 10 makes it possible to compress the dry steam. The aspiration device 2 also comprises an aspiration straw 12 opening out into the aspiration cone 10. The aspiration straw 12 is also used for cooling. The aspiration device 2 is made, for example, from a medical stainless steel or any other material intended for medical use.

In order to protect the user and avoid burns during handling of the inhaler, a thermal insulator (not shown) may be provided arranged around the heating element 1. This thermal insulator may be present in the form of a ring which is fixed under the heating element 1, so as not to obstruct the ends 6a of the air aspiration channels 6.

The inhaler according to the invention has a very solid and economical design, allowing simple use. These reduced dimensions make it a pocket-format inhaler, easily transportable, while also being optimized to ensure sufficient heating time without excessive or periodic heating and minimizing the risk of combustion. The materials used allow easy washing and sterilization of the inhaler.

During use of the inhaler according to the invention, the substance is placed in the aspiration chamber of the heating element, which is then closed by screwing the aspiration device. Then the inhaler is heated using a flame, for example using a lighter with a so-called “blowpipe” or “laser” flame, or another adapted heat source, placed under the heating crucible. The duration of the heating is approximately 30 to 60 seconds in order, during the first aspiration, to bring the substance to the extraction temperature of the active principle, then approximately 10 seconds for the following aspirations. The form of the heating element and the choice of a heat conducting material cause the heat to spread rapidly in the heating element, which serves as a heat reservoir, without periodic overheating. Then, when the user inhales air using the aspiration device, the air penetrates the various air aspiration channels. The aspirated air circulates in the air aspiration channels which extend between the side wall and the combustion chamber, itself heating via heat conduction by the heating element. The aspirated air then makes a sufficient journey to reheat itself while traversing the heating element and therefore arrives hot on the substance placed in the inhaler, without cooling it, which enables optimal activation of its extraction. The thermal inertia of the material of the heating element allows prolonged use and therefore several successive long aspirations without proceeding with a new heating. One will note that the heating element may comprise a smaller number of channels, but with a larger diameter so as to preserve a sufficient draw.

1-10. (canceled)

11. A dry-steam inhaler, comprising a heating element and an aspiration device, wherein the heating element comprises a base and side walls which project from said base and delimit an aspiration chamber communicating with the aspiration device, the base of the heating element being traversed by at least one air aspiration channel whereof one end opens out on the side walls of the heating element and the other end of which opens out in the aspiration chamber.

12. The inhaler of claim 11, wherein the base of the heating element comprises four air aspiration channels arranged in a cross, their ends which open out in the aspiration chamber meeting at the center of said aspiration chamber.

13. The inhaler of claim 11, wherein the air aspiration channels are arranged in the base of the heating element parallel to said base.

14. The inhaler of claim 12, wherein the air aspiration channels are arranged in the base of the heating element parallel to said base.

15. The inhaler of claim 11, wherein the side walls of the heating element comprise a lower extension projecting from the bottom opposite the aspiration chamber and delimiting a heating crucible with said base.

16. The inhaler according to claim 15, wherein the air aspiration channel has a diameter smaller than or equal to 2 mm and is arranged in the base of the heating element parallel to the heating crucible and 1 mm or less from it, and parallel to the aspiration chamber and 1 mm or less from it.

17. The inhaler of claim 11, wherein the heating element is cylindrical in shape.

18. The inhaler of claim 11, wherein the base of the heating element comprises, in its center, a hollow form into which the ends of the air aspiration channels open.

19. The inhaler of claim 11, wherein the heating element is made in an aluminum-based material.

20. The inhaler according to claim 11, wherein the aspiration device comprises an aspiration cone arranged to cooperate with the heating element and an aspiration straw.

21. The inhaler according to claim 11, characterized in that it comprises a thermal insulator arranged around the heating element.
